(19) United States
(12) Patent Application Publication
(10) Pub. No.: US 2024/0215964 A1
(43) Pub. Date: Jul. 4, 2024

(54) POWERED HANDLE ASSEMBLY FOR SURGICAL DEVICES

(71) Applicant: Covidien LP, Mansfield, MA (US)

(72) Inventor: David A. Nicholas, Trumbull, CT (US)

(21) Appl. No.: 18/609,145

(22) Filed: Mar. 19, 2024

Related U.S. Application Data
(63) Continuation of application No. 17/224,669, filed on Apr. 7, 2021, now Pat. No. 11,937,794.
(60) Provisional application No. 63/022,979, filed on May 11, 2020.

(51) Int. Cl. A61B 17/00; A61B 17/072

(52) U.S. Cl. CPC A61B 17/00; A61B 17/072; A61B 2017/00398; A61B 2017/00424; A61B 2017/00477; A61B 2017/00734

(57) ABSTRACT

A surgical device with a powered in-line handle assembly that includes actuators for actuating and articulating an end effector of the surgical device allows for single handed operation of the surgical device. The handle assembly includes a battery that is received in a removable compartment in a battery cover to allow for removal and recycling of the battery following use. The handle assembly also includes a manual release mechanism that allows a firing rod assembly of the handle assembly to be manually retracted in emergency situations.

### CROSS-REFERENCE TO RELATED APPLICATIONS

This application is a continuation of U.S. patent application Ser. No. 17/224,669 filed Apr. 7, 2021 and claims the benefit of and priority to U.S. Provisional Patent Application No. 63/022,979, filed May 11, 2020, the entire contents each of which are incorporated by reference herein.

### FIELD

This technology is generally related to handle assemblies and, more particularly, to in-line handle assemblies for powered surgical stapling devices.

### BACKGROUND

Surgical devices are commonly used during surgical procedures to perform a variety of different surgical operations including stapling, grasping, cutting, and sealing tissue to name a few. Typically, a surgical device includes a handle assembly that a clinician, e.g., a surgeon, grasps to actuate the surgical device. Certain types of surgical devices perform multiple tasks and have different capabilities to allow the device to more easily access tissue within a body cavity. For example, powered surgical stapling devices include actuation buttons for approximating jaws of a tool assembly of the stapling device, for applying staples to tissue, and for cutting tissue. These stapling devices can also include knobs to facilitate articulation and/or rotation of the tool assembly in relation to the handle assembly.

During a surgical procedure, the clinician may have to operate the stapling device while simultaneously performing a second task, e.g., stabilizing the tissue. During such procedures, it would be advantageous if the stapling device included a handle assembly that a clinician could operate using a single hand to free up the clinician's other hand to perform other operations.

### SUMMARY

In aspects, this disclosure relates generally to a surgical device including a powered in-line handle assembly that includes actuators for actuating and articulating an end effector of the surgical device to allow for single handed operation of the surgical device. The actuators are positioned on the handle assembly in close relation to each other to be accessible with a gripping hand of the clinician. The handle assembly includes a battery that is received in a removable compartment in a battery cover to allow for removal and recycling of the battery following use. The handle assembly also includes a manual release mechanism that allows a firing rod assembly of the handle assembly to be manually retracted in emergency situations.

One aspect of the disclosure is directed to a handle assembly for a surgical device that includes a stationary body, a distal body portion, a firing rod assembly, a motor, a release member, and a battery cover. The stationary body includes a proximal portion and a distal portion and defines a cavity. The distal and proximal portions of the stationary body each define an opening. The stationary body has an outer gripping surface. The distal body portion extends distally from the stationary body through the opening in the distal portion of the stationary body. The distal body portion defines a longitudinal axis and the gripping surface of the stationary body is positioned about the longitudinal axis. The firing rod assembly includes a firing rod that extends within the distal body portion and a drive rod. The drive rod is supported within the cavity of the stationary body and has a distal portion coupled to the firing rod and a proximal portion. The motor includes a drive shaft supported within the cavity of the stationary body. The motor is coupled to the drive rod and is operable to move the drive rod between a retracted position and an advanced position. Movement of the drive rod between its retracted and advanced positions moves the firing rod between retracted and advanced positions. The release member is attached to the proximal portion of the drive rod and extends through the proximal portion of the stationary body. The battery cover is supported on the proximal portion of the stationary body and is movable between an open position and a closed position. The release member extends into the battery cover when the battery cover is in the closed position and is accessible when the battery cover is in its open position.

In aspects of the disclosure, the release member supports a transverse member and the battery shell defines guide slots that receive the transverse member to guide the battery cover as the battery cover moves from the open position towards the closed position.

Another aspect of the disclosure is directed to a surgical stapling device including a handle assembly and an end effector. The handle assembly includes a stationary body, a distal body portion, a firing rod assembly, a motor, a release member, and a battery cover. The stationary body includes a proximal portion and a distal portion and defines a cavity. The distal and proximal portions of the stationary body each define an opening. The stationary body has an outer gripping surface. The distal body portion extends distally from the stationary body through the opening in the distal portion of the stationary body. The distal body portion defines a longitudinal axis and the gripping surface of the stationary body is positioned about the longitudinal axis. The firing rod assembly includes a firing rod that extends within the distal body portion and a drive rod. The drive rod is supported within the cavity of the stationary body and has a distal portion coupled to the firing rod and a proximal portion. The motor includes a drive shaft supported within the cavity of the stationary body. The motor is coupled to the drive rod and is operable to move the drive rod between a retracted position and an advanced position. Movement of the drive rod between its retracted and advanced positions moves the firing rod between retracted and advanced positions. The release member is attached to the proximal portion of the drive rod and extends through the proximal portion of the stationary body. The battery cover is supported on the proximal portion of the stationary body and is movable between an open position and a closed position. The release member extends into the battery cover when the battery cover is in the closed position and is accessible when the battery cover is in its open position. The end effector is supported on the distal body portion of the handle assembly and includes an anvil assembly and a cartridge assembly. The anvil and cartridge assemblies are movable in relation to each other between open and clamped positions.

In aspects of the disclosure, the firing rod assembly includes a connector that is fixedly coupled to the drive rod and is rotatably coupled to the firing rod.

In some aspects of the disclosure, a threaded firing nut is coupled to the motor, and the drive rod is threaded and is engaged with the threaded firing nut such that rotation of the threaded firing nut in response to actuation of the motor causes longitudinal movement of the drive rod between its retracted and advanced positions.

In certain aspects of the disclosure, the motor is coupled to the threaded firing nut by a spur gear and a firing nut gear. The spur gear is secured to the drive shaft of the motor and the firing nut gear is secured to the threaded firing nut.

In aspects of the disclosure, the release member is fixedly secured to the proximal portion of the drive rod.

In some aspects of the disclosure, the release member includes a proximal portion that supports a transverse shaft that is positioned proximally of the stationary body.

In certain aspects of the disclosure, an articulation assembly includes an articulation knob that is rotatable about the distal portion of the stationary body and about the longitudinal axis.

In aspects of the disclosure, the articulation assembly includes an articulation nut that is fixedly secured to the articulation knob, and an articulation screw.

In some aspects of the disclosure, the articulation nut includes internal threads and the articulation screw includes external threads that are engaged with the internal threads of the articulation nut such that rotation of the articulation knob causes rotation of the articulation nut to effect longitudinal movement of the articulation screw.

In certain aspects of the disclosure, the articulation assembly includes an articulation link that is coupled to the articulation screw and is supported within the distal body portion of the handle assembly for longitudinal movement between advanced and retracted positions.

In aspects of the disclosure, the articulation nut includes longitudinal ribs and the articulation knob defines internal slots that receive the longitudinal ribs to secure the articulation knob to the articulation nut.

In some aspects of the disclosure, the stationary body supports at least one actuation button for controlling operation of the motor.

In certain aspects of the disclosure, a printed circuit board is supported within the stationary body and includes a processor that is electrically connected to the motor and the at least one actuation button.

In aspects of the disclosure, the printed circuit board includes a rotary encoder that measures speed and direction of the drive shaft of the motor.

In some aspects of the disclosure, a USB port is coupled to the printed circuit board.

In certain aspects of the disclosure, the distal body portion has a distal portion that is adapted to releasably engage an end effector.

In aspects of the disclosure, the battery cover is pivotably supported on the proximal portion of the stationary body between the open and closed positions.

In some aspects of the disclosure, a battery shell is releasably coupled to the battery cover and defines a cavity that is configured to receive a battery.

In aspects of the disclosure, the end effector forms part of a reload assembly that is releasably coupled to the distal body portion of the handle assembly.

Other features of the disclosure will be appreciated from the following description.

### BRIEF DESCRIPTION OF DRAWINGS

Various aspects of a handle assembly according to the disclosure are described herein below with reference to the drawings, wherein:

FIG. 1 is a side perspective view from a distal end of the disclosed powered handle assembly;

FIG. 2 is a side perspective view from a proximal end of the powered handle assembly shown in FIG. 1;

FIG. 3 is an exploded view of the powered handle assembly shown in FIG. 1;

FIG. 4 is a side perspective view of the powered handle assembly shown in FIG. 2 with a body half-section removed;

FIG. 5 is a side perspective view of the powered handle assembly shown in FIG. 2 with the body half-section removed, an outer tube removed, and a rotation knob removed;

FIG. 6 is a cross-sectional view taken along section line 6-6 of FIG. 1;

FIG. 7 is an enlarged view of the indicated area of detail shown in FIG. 6;

FIG. 8 is a cross-sectional view taken along section line 8-8 of FIG. 6;

FIG. 9 is an enlarged view of the indicated area of detail shown in FIG. 8;

FIG. 10 is a cross-sectional view taken along section line 10-10 of FIG. 1;

FIG. 11 is a cross-sectional view taken along section line 11-11 of FIG. 1;

FIG. 12 is a cross-sectional view taken along section line 12-12 of FIG. 6;

FIG. 13 is a side perspective view of a proximal portion of the handle assembly shown in FIG. 1 with the body half-section and the battery cover removed;

FIG. 14 is a side perspective view of the proximal portion of the handle assembly shown in FIG. 1 with the battery cover in an open position;

FIG. 15 is a cross-sectional view taken along section line 15-15 of FIG. 14; and

FIG. 16 is a cross-sectional view taken through the battery cover and battery of the powered handle assembly shown in FIG. 1.

### DETAILED DESCRIPTION

The disclosed surgical device will now be described in detail with reference to the drawings in which like reference numerals designate identical or corresponding elements in each of the several views. However, it is to be understood that the aspects of the disclosure are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure. In addition, directional terms such as front, rear, upper, lower, top, bottom, distal, proximal, and similar terms are used to assist in understanding the description and are not intended to limit the disclosure.

In this description, the term “proximal” is used generally to refer to that portion of the device that is closer to a clinician, while the term “distal” is used generally to refer to that portion of the device that is farther from the clinician. Further, the term “clinician” is used generally to refer to medical personnel including doctors, nurses, and support personnel.

This disclosure is directed to a surgical device including a powered in-line handle assembly that includes actuators for actuating and articulating an end effector of the surgical device to allow for single handed operation of the surgical device. The actuators are positioned on the handle assembly in close relation to each other to be accessible with a gripping hand of the clinician. The in-line handle assembly is received in the palm of a clinician's hands and allows a clinician to rotate the end effector of the surgical device by simply rotating the clinician's wrist thus obviating the need for additional rotation mechanisms. A battery is received in a removable compartment in a battery cover to allow for removal and recycling of the battery following use. The handle assembly includes a manual release mechanism that allows a firing rod assembly of the handle assembly to be manually retracted in emergency situations.

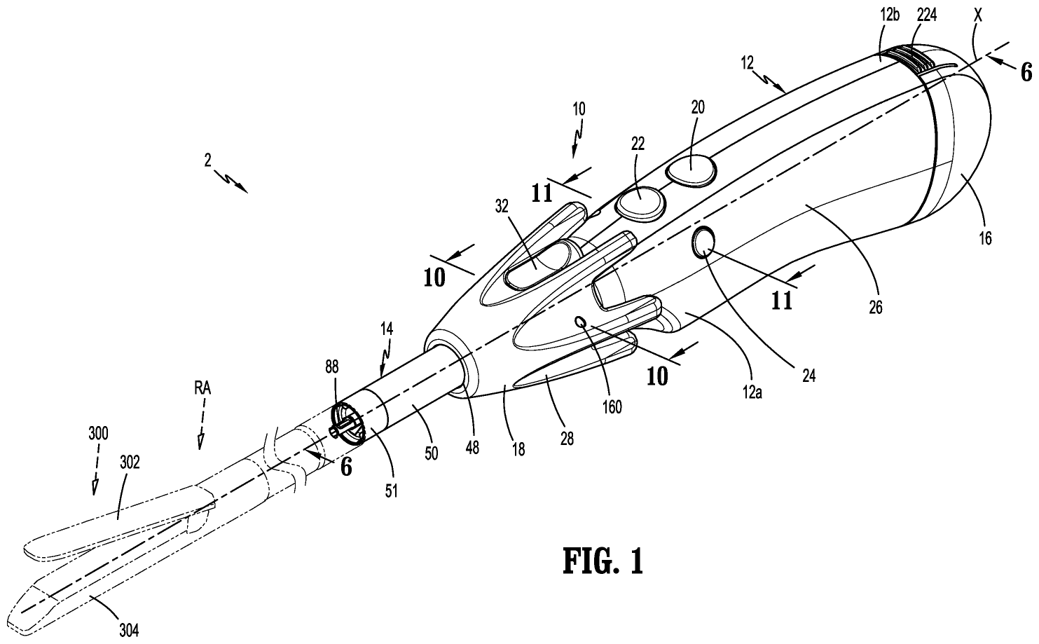

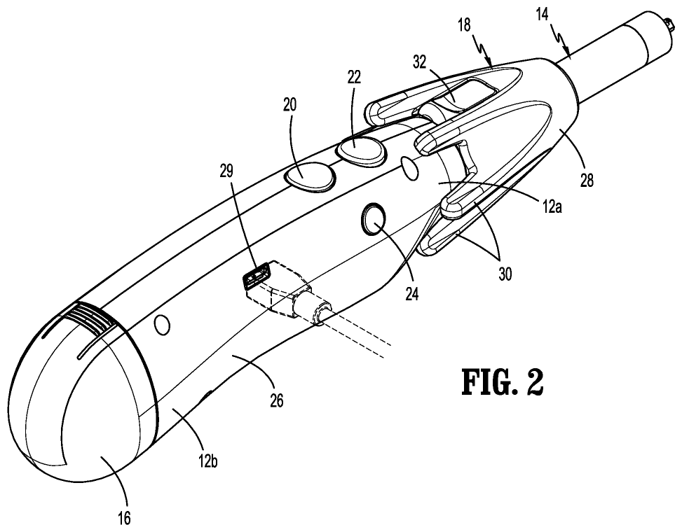

FIGS. 1 and 2 illustrate aspects of the disclosed powered handle assembly for a surgical stapling device 2 which is shown generally as handle assembly 10. It is envisioned that the handle assembly 10 can be used with a variety of different types of surgical devices other than stapling devices. The handle assembly 10 includes a stationary body 12 and a distal body portion 14 that extends from a distal portion 12a of the stationary body 12 and defines a longitudinal axis “X”. The stationary body 12 includes a proximal portion 12b that supports a battery cover 16 that is movable between a closed position (FIG. 1) and an open position (FIG. 14) to facilitate placement of a battery or batteries into the powered handle assembly 10. The distal portion 12a of the stationary body 12 supports an articulation knob 18 that is manually operable, e.g., by action of the clinician's fingers, to control articulation of an end effector 300 of a reload assembly “RA” that is supported on the distal body portion 14 of the handle assembly 10. The reload assembly “RA” including the end effector 300 is shown in phantom in FIG. 1. In aspects of the disclosure, the reload assembly “RA” is coupled to the distal portion 12a of the stationary body 12 of the handle assembly 10 and the end effector 300 can pivot in response to actuation of the articulation knob 18 about an axis transverse to the longitudinal axis “X”. The stationary body 12 of the handle assembly 10 also includes a plurality of actuation buttons to operate various functions of the end effector 300, e.g., clamping, stapling, and cutting. In some aspects of the disclosure, the stationary body 12 supports a first actuation button 20 that controls opening of the end effector 300 and a second actuation button 22 that controls clamping and firing of the end effector 300 wherein firing includes stapling and/or cutting of tissue. In some aspects of the disclosure, a safety button 24 is supported on each side of the stationary body 12 of the handle assembly 10. In aspects of the disclosure, the safety button 24 must be depressed or actuated before the actuation button 22 can be operated to fire the end effector 300.

In aspects of the disclosure, the end effector 300 includes an anvil assembly 302 and a cartridge assembly 304. The anvil assembly 302 and the cartridge assembly 304 are coupled together for movement between an open position and a clamped position to clamp tissue between the anvil and cartridge assemblies 302 and 304, respectively. The cartridge assembly 304 supports a plurality of staples (not shown) that are supported in pockets on opposite sides of a knife slot (not shown). The end effector includes a drive member (not shown) that is movable in relation to the anvil and cartridge assemblies 302 and 304 to move the anvil and cartridge assemblies to the clamped position to eject staples from the cartridge assembly 304 and/or cut tissue. For a more detailed description of an exemplary end effector including anvil and cartridge assemblies, see U.S. Pat. No. 10,123,799 (“the '799 Patent”).

It is envisioned that the end effector 300 can be permanently attached to the distal body portion 14 of the handle assembly 10 and need not form part of a releasable reload assembly.

The stationary body 12 extends along an axis that is substantially aligned with the longitudinal axis “X” of the distal body portion 14 of the handle assembly 10. In aspects of the disclosure, the stationary body 12 of the handle assembly 10 includes a gripping surface 26 that is positioned about the longitudinal axis “X” and is ergonomically configured to be gripped by a clinician. In that respect, the stationary body 12 can be slightly curved along the longitudinal axis “X” and/or include grooves to receive one or more of a clinician's fingers. The actuation buttons 20 and 22, the safety button 24, and the articulation knob 18 are all supported in close relation to each other on the stationary body 12 to facilitate single handed operation of the handle assembly 10 by the clinician. The stationary body 12 includes a side wall that defines an USB port 29 that is described in further detail below.

In some aspects of the disclosure, the articulation knob 18 is rotatably supported about the distal portion 12a of the stationary body 12 and includes a scalloped body 28 that includes proximally extending fingers 30. The proximally extending fingers 30 extend proximally towards the actuation buttons 20 and 22 and towards the safety buttons 24 to facilitate single handed operation of the handle assembly 12. In some aspects of the disclosure, the articulation knob 18 supports a reload release button 32 which is described in further detail below. In certain aspects of the disclosure, the distance between the proximal-most button 20, 22 and 24 and the proximal end of the fingers 30 of the articulation knob 18 is from about 1 inch to about 3 inches and may be about 2 inches. In other aspects of the disclosure, the distance between the proximal-most button 20, 22 and 24 and the proximal end of the fingers 30 is less than 3 inches.

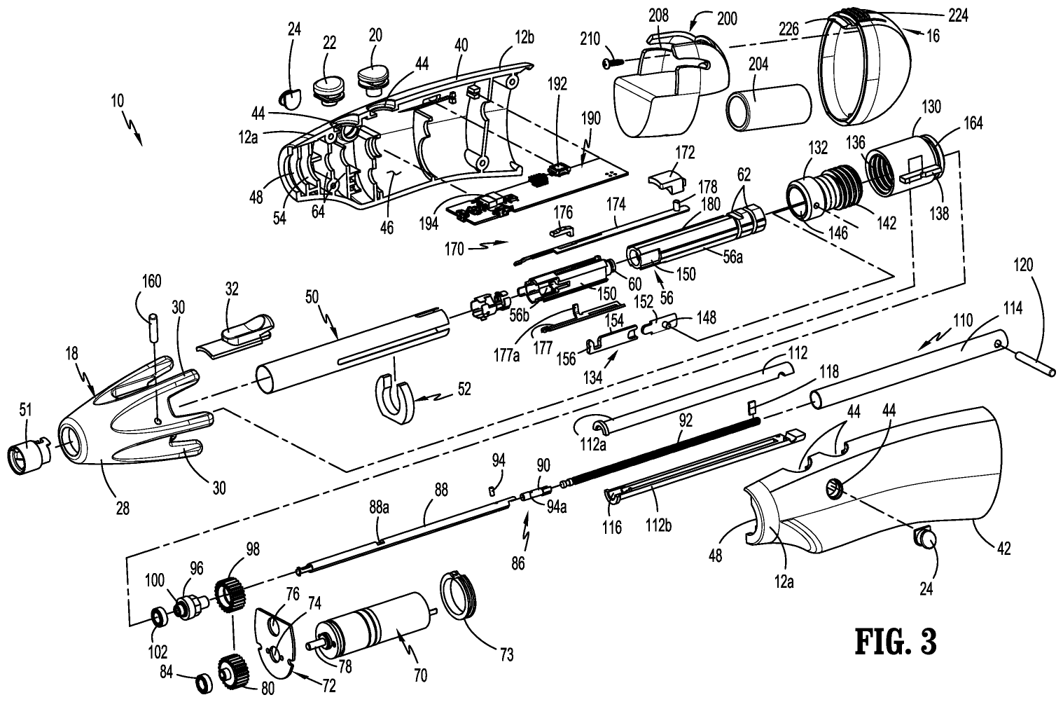

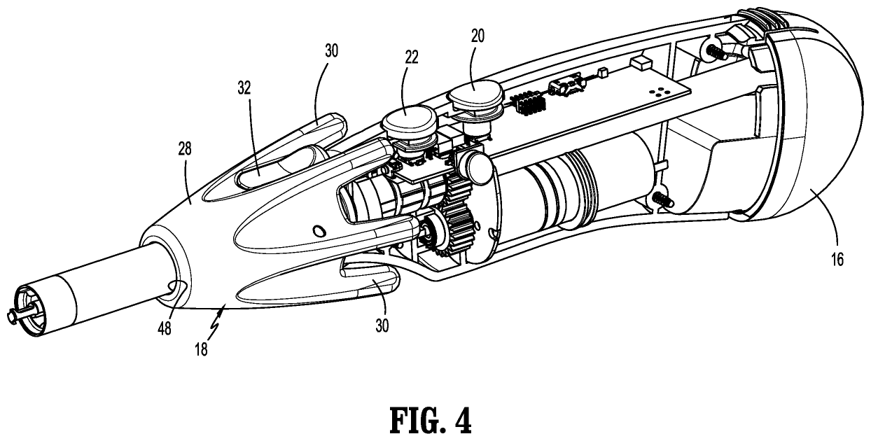

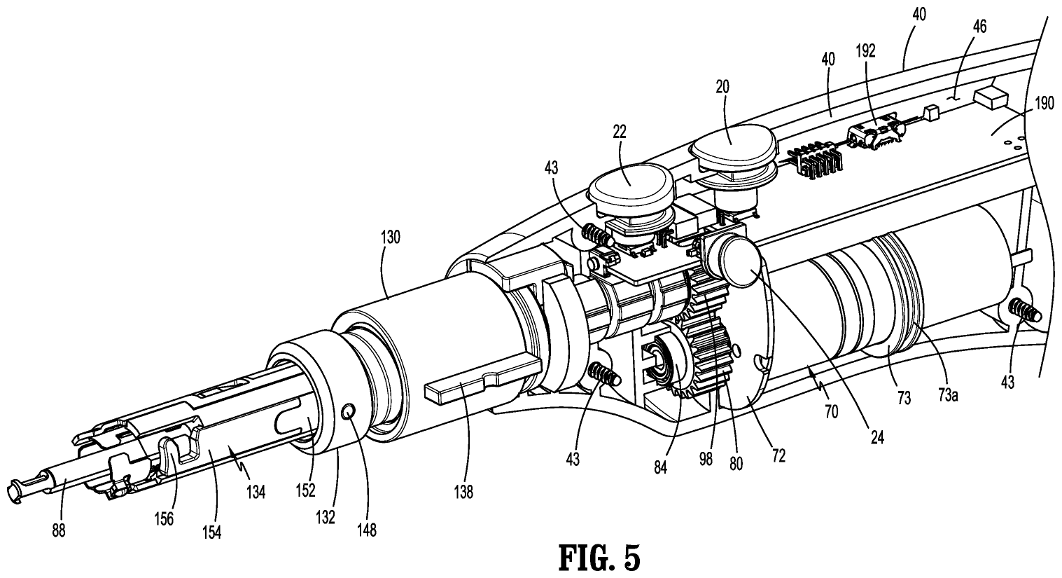

FIGS. 3-12 illustrate the internal components of the handle assembly 10. The stationary body 12 of the handle assembly 10 is formed from molded half sections 40 and 42 that are secured together using, e.g., screws 43 (FIG. 5) or welding, to define a cavity 46 within the stationary handle 12. Each of the half-sections 40 and 42 of the stationary body 12 defines openings or portions of openings 44 that receive the actuation buttons 20 and 22 and the safety buttons 24 of the handle assembly 10. The distal portion 12a of the defines an opening 48 that receives the distal body portion 14 of the handle assembly 10.

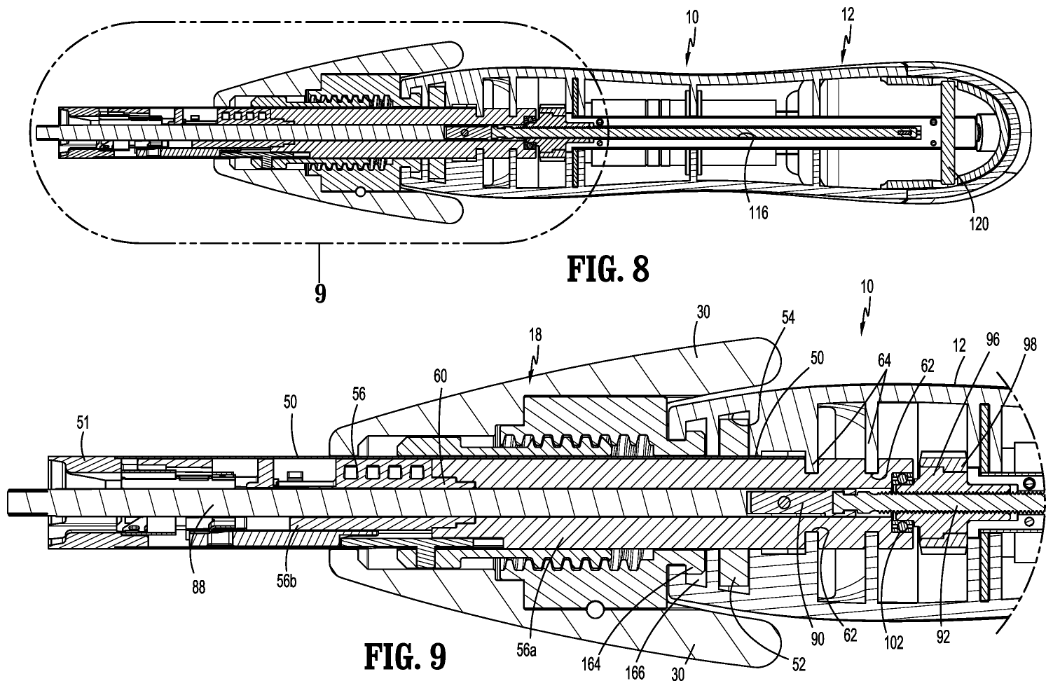

The distal body portion 14 of the handle assembly 10 includes an outer tube 50 that extends through the opening 48 in the distal portion 12a of the stationary body 12 and communicates with the cavity 46. The outer tube 50 is welded to a collar 52 and the collar is received within a slot 54 (FIG. 9) defined between the half-sections 40 and 42 of the stationary body 12 to secure the outer tube 50 within the stationary body 12. The distal end of the outer tube 50 receives a member 51 that defines internals channels (not shown) that direct the reload assembly “RA” (FIG. 1) into engagement with the distal body portion 14 of the handle assembly 10 in bayonet-type fashion.

The distal body portion 14 of the handle assembly 10 includes a housing 56 that is fixedly retained within the outer tube 50. In aspects of the disclosure, the housing 56 includes a proximal housing 56a (FIG. 3) and a distal housing 56b that define a through bore 58 (FIG. 7), The distal housing 56b includes an extension 60 (FIG. 3) that is received within the distal end of the proximal housing 56a. In aspects of the disclosure, the proximal housing 56a includes annular slots 62 (FIG. 3) that receive ribs 64 (FIG. 9) formed on inner walls of the half-sections 40 and 42 of the stationary body 12 to secure the housing 56 within the outer tube 50. The housing 56 including the proximal and distal housing 56a and 56b define channels within the distal body portion 14 that guide movement of internal components of the handle assembly 10 as described in further detail below.

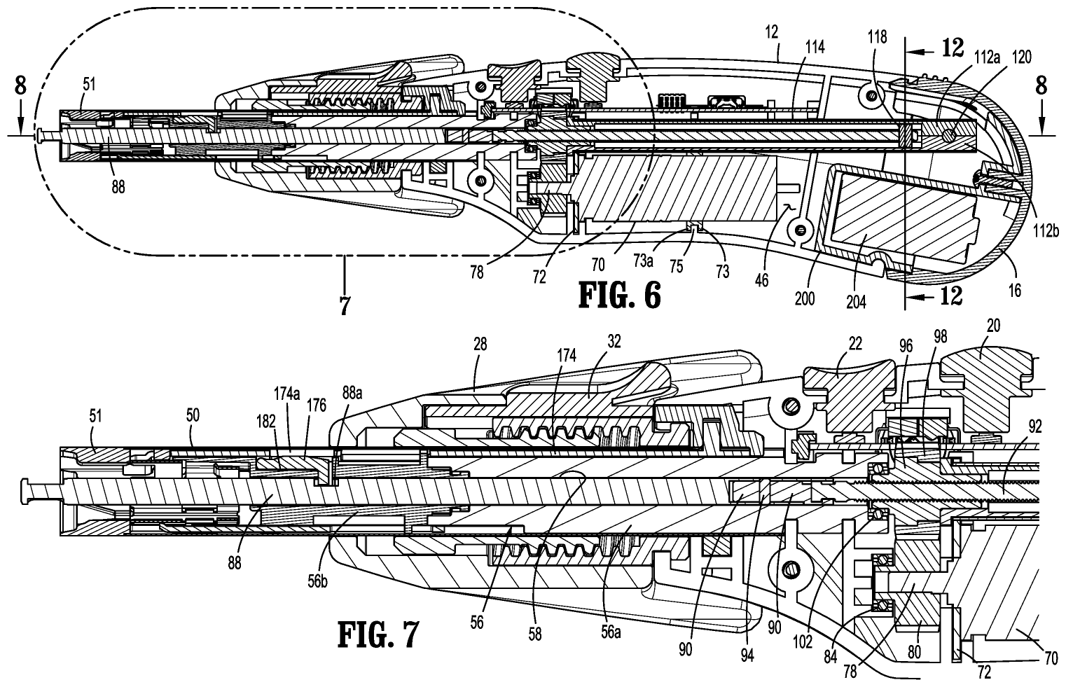

The handle assembly supports a motor 70 that is received within the cavity 46 of the stationary body 12. The motor 70 is secured to a proximal side of a mounting plate 72 that is secured within the cavity 46 of the stationary body 12 by screws or the like. The motor 70 is received within a rubber motor vibration damper 73 that is supported within the cavity 46 of the stationary body 12. In aspects of the disclosure, the vibration damper 73 defines a central channel 73a (FIG. 5) that receives a rib 75 (FIG. 6). The mounting plate 72 includes a first bore 74 (FIG. 3) and a second bore 76. The drive shaft 78 of the motor 70 extends through the first bore 74 of the mounting plate 72 and is secured to a spur gear 80. The spur gear 80 defines a D-shaped bore 82 (FIG. 11) and the drive shaft 78 is D-shaped such that rotation of the drive shaft 78 causes corresponding rotation of the spur gear 80. The distal end of the spur gear 80 and drive shaft 78 are supported within the stationary body 12 by a bearing 84.

A firing rod assembly 86 (FIG. 3) is supported within the stationary body 12 of the handle assembly 10. The firing rod assembly 86 includes a firing rod 88, a connector 90, and a threaded drive rod 92. The firing rod 88 includes a notch 88a (FIG. 3). The connector 90 defines a transverse bore 94a (FIG. 3) and is fixedly coupled to a proximal end of the firing rod 88 by a pin 94 that is received within the transverse bore 94a. The distal end of the drive rod 92 is rotatably coupled to the connector 90 such that axial movement of the drive rod 92 causes axial movement of the connector 90 and axial movement of the firing rod 88.

The distal end of the drive rod 92 is received within a threaded firing nut 96 (FIG. 7) and the threaded firing nut 96 is secured within a firing nut gear 98 such that rotation of the firing nut gear 98 causes rotation of the firing nut 96. The firing nut gear 98 and the firing nut 96 are axially fixed within the stationary body 12 of the handle assembly 10. In aspects of the disclosure, the firing nut 96 has an octagonal outer configuration (FIG. 11) and the inner surface of the firing nut gear 98 has an inner octagonal configuration that is engaged with the outer octagonal configuration of the firing nut 96 to fixedly secure the firing nut gear 98 to the firing nut 96. In aspects of the disclosure, the distal end of the firing nut 96 defines a hub 100 (FIG. 3) that is supported on a bearing 102.

As described above, the firing nut 96 is threaded and is engaged with the threaded drive rod 92 of the firing rod assembly 86. When the motor 70 is actuated to rotate the drive shaft 78, the drive shaft 78 which is secured to the spur gear 80 rotates the spur gear 80. The spur gear 80 is engaged with the firing nut gear 96 such that rotation of the spur gear 80 causes rotation of the firing nut gear 98. When the firing nut gear 98 rotates, the firing nut 96 rotates with the firing nut gear 98. Since the firing nut 96 is axially fixed within the stationary body 12 of the handle assembly 10, rotation of the firing nut 96 in relation to the threaded drive rod 92 causes longitudinal movement of the firing rod 96 which in turn causes corresponding axial movement of the firing rod 88 through the distal body portion 14 of the handle assembly 10. The distal end of the firing rod 88 is configured to engage a drive member (not shown) of the reload assembly “RA” (FIG. 1). The '799 Patent discloses a handle assembly and a reload assembly that is releasably coupled to the handle assembly that includes a firing rod that is releasably coupled to a drive member of the reload assembly.

The handle assembly 10 includes a manual retraction mechanism 110 (FIG. 3) that is accessible through the proximal portion 12b (FIG. 14) of the stationary body 12 when the battery cover 16 is opened to facilitate manual retraction the firing rod 88 if the powered handle assembly 10 becomes inoperable. The manual retraction mechanism 110 includes a release housing 112 and a release member or tube 114 that are received about the threaded drive rod 92. The release housing 112 is formed from two half-sections 112a and 112b that define a longitudinal through bore 116. The release housing 112 is received within the release tube 114 to prevent separation of the half-sections 112a and 112b (FIG. 3) of the release housing 112. The drive rod 92 is fixedly coupled to the release housing 112 by a key 118 (FIG. 6). The release housing 112 is coupled to the release tube 114 by a transverse pin or shaft 120. The transverse pin 120 extends through an opening 122 in the proximal end of the release tube 114 and through an opening 124 in the proximal end of the release housing 112 to fixedly couple the release tube 114 and to the release housing 112. When the release tube 114 is rotated by turning the transverse pin 120, the release housing 112 and the drive rod 92 are rotated within the stationary body 12 of the handle assembly 10. As the drive rod 92 rotates in relation to the threaded firing nut 96, the drive rod 92 and the firing rod 88 are moved longitudinally within the handle assembly 10. The drive rod will move proximally or distally depending on the direction of rotation of the firing nut 96.

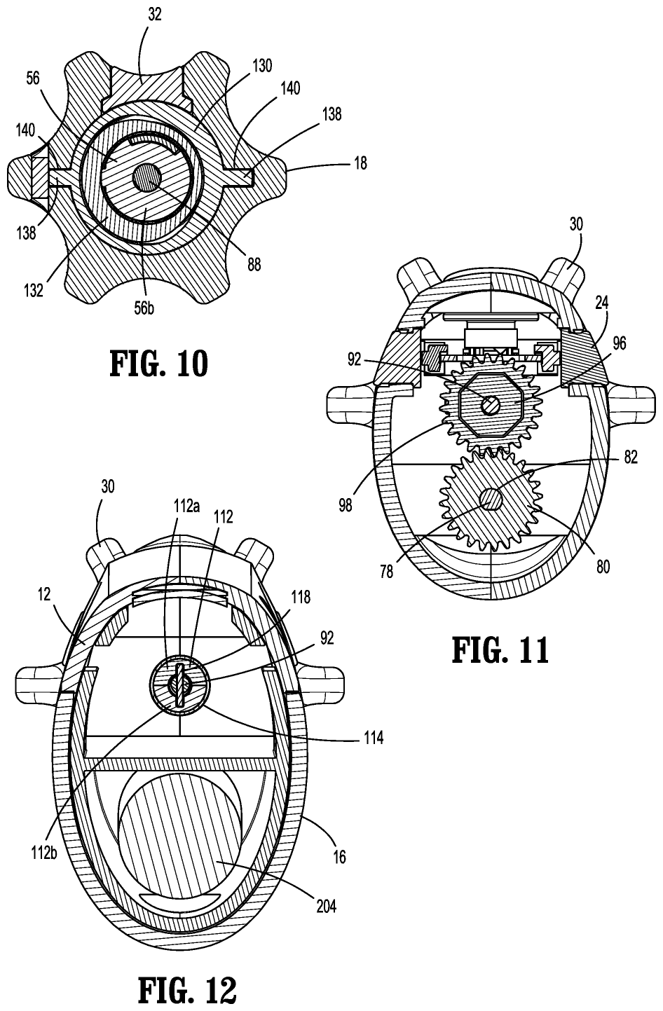

The articulation knob 18 forms part of an articulation assembly that includes an articulation nut 130, an articulation screw 132, and an articulation link 134. The articulation nut 130 defines a threaded longitudinal through bore 136 (FIG. 3) and includes longitudinal ribs 138 (FIG. 10). The longitudinal ribs 138 are received within slots 140 (FIG. 10) defined within an inner surface of the articulation knob 18. The articulation nut 130 is received about the proximal housing 56a (FIG. 3) of the housing 56 of the distal body portion 14 (FIG. 1) of the handle assembly 10. The articulation screw 132 includes an outer threaded surface 142 and defines an inner longitudinal through bore 146 (FIG. 3). The articulation screw 132 is received within the threaded through bore 136 of the articulation nut 130.

The articulation link 134 is secured to the inner surface of the articulation screw 132 by a pin 148 (FIG. 3) which is fixed to and extends radially outward from the articulation link 134. The articulation link 134 is slidably positioned within a longitudinal recess 150 that is defined within an outer surface of the housing 56 of the distal body portion 14 of the handle assembly 10. The longitudinal recess 150 (FIG. 3) limits the articulation link 134 to longitudinal movement within the outer tube 50 of the distal body portion 14 of the handle assembly 10. In aspects of the disclosure, the articulation link 134 can be formed from a proximal link portion 152 and a distal link portion 154. Alternately, the articulation link 134 can be formed of unitary construction. The articulation link 134 includes a distal hook 156 (FIG. 5) that is configured to releasably engage an articulation link (not shown) in the reload assembly “RA” (FIG. 1) when the reload assembly “RA” is coupled to the distal body portion 14 of the handle assembly 10.

The articulation knob 18 is coupled to the articulation nut 130 by a pin 160 (FIG. 3). The articulation nut 130 includes a proximal flange 164 (FIG. 9) that is received within an annular recess 166 defined within the stationary body 12 of the handle assembly 10 and facilitates rotation of the articulation nut 130 in relation to the stationary body 12 of the handle assembly 110. Receipt of the proximal flange 164 of the articulation nut 130 within the annular recess 166 of the stationary body 112 prevents longitudinal movement of the articulation nut 130 in relation to the stationary body 12.

When the articulation knob 18 is rotated about the longitudinal axis “X” (FIG. 1) by a clinician, the articulation nut 130 which is pinned to the articulation knob 18 is also rotated about the longitudinal axis “X”. When the articulation nut 130 rotates in relation to the stationary body 112, the threads defining the through bore 136 of the articulation nut 130 engage the threads on the threaded surface 142 of the articulation screw 132 and move the articulation screw 132 longitudinally within the stationary body 112 of the handle assembly 110. As the articulation screw 132 moves longitudinally within the stationary body 112 of the handle assembly 12, the articulation link 134 moves longitudinally within the outer tube 50 of the distal body portion 14 (FIG. 1) of the handle assembly 10. As stated above, the distal portion of the articulation link 134 includes a hook 156 that engages an articulation link (not shown) in the reload assembly “RA” (FIG. 1) such that longitudinal movement of the articulation link 134 causes the end effector 300 of the reload assembly “RA” to articulate about an axis transverse to the longitudinal “X” (FIG. 1) of the distal body portion 114 of the handle assembly 110. The '799 Patent discloses a reload assembly including an articulation link that is configured to be releasably coupled to the articulation link of an adapter assembly in a manner like that described herein.

The handle assembly 10 also includes a lock mechanism 170 (FIG. 3) that fixes the axial and radial orientation of the firing rod 88 when a reload assembly “RA” is coupled to the handle assembly 10. The lockout assembly 170 would not be included in a surgical stapling device 2 in which the end effector 300 is fixedly coupled to the distal body portion of the handle assembly 10. In aspects of the disclosure, the lock mechanism 170 includes the release button 32, a connector 172, a release link 174, a lockout member 176, and a reload detect link 177 (FIG. 3). The release button 32 is slidably supported on the articulation knob 18 between two adjacent fingers 30 of the articulation knob 30 and is engaged with the connector 172. The connector 172 is coupled to the release link 174 by a post 178 (FIG. 3). The release link 174 is slidably supported in a channel 180 defined in the housing 56 (FIG. 3) of the distal body portion 14 of the handle assembly 12. The lockout member 176 is supported in a recess 182 (FIG. 7) in the housing 56 and extends through a slot 174 in the release link 174. The lockout member 176 is pivotable between a position spaced from the notch 88a in the firing rod 88 to a position received within the notch 88a of the firing rod 88. The reload detect link 177 includes a distal end that extends into the distal end of the distal body portion 14 of the handle assembly 10 and a finger 177a that is engaged with the release link 174.

As stated above, a reload assembly “RA” is coupled to the distal body portion 14 of the handle assembly 10 by inserting a proximal end of the reload assembly “RA” into the distal end of the distal body portion 14 and subsequently rotating the reload assembly “RA” in relation to the distal body portion 14 to form a bayonet type coupling. When the reload assembly “RA” is inserted into the distal body portion 14, the reload assembly “RA” engages the distal end of the reload detect link 177 to move the reload detect link 177 proximally. The reload detect link 177 engages the release link 174 and moves the release link 174 proximally within the distal body portion 14. When the release link 174 moves proximally, the release link 174 engages and cams the lockout member 176 against the force of a biasing member (not shown) to its locked position received within the notch 88a in the firing rod 88.

When the reload assembly “RA” is rotated in relation to the distal body portion 14 of the handle assembly 10, to couple the reload assembly “RA” to the distal body portion 14, a biasing member not shown moves the release link 174 distally to a position locking the reload assembly “RA” onto the distal body portion 14 of the handle assembly 10, i.e., blocking rotation of the reload assembly “RA” in relation to the distal body portion 14 of the handle assembly 10. In order to release the reload assembly “RA” from the distal body portion 14 of the handle assembly 10 after the reload assembly “RA” is fired, the release button must be moved proximally to move the release link to a position to allow the reload to be rotated to uncouple the reload assembly “RA” from the distal body portion 14. For a more detailed description of the operation of the lockout mechanism 170, see the '799 Patent.

The handle assembly 10 supports a printed circuit board (PCB) 190 (FIG. 5) which is received within the cavity 46 of the stationary body 12 and is electrically coupled to the motor 70, the actuation buttons 20 and 22, and the safety buttons 24. The PCB 190 accommodates a motor controller, switches that are coupled to the actuation and safety buttons 20, 22, and 24 (FIG. 3), and a processor that controls operation of the handle assembly 10 to control actuation of the end effector 300 (FIG. 1). In some aspects of the disclosure, a USB socket 192 is connected to the PCB 190 and can be used to communicate with the processor to access, e.g., load code. The USB socket 192 is accessible through the port 29 (FIG. 2) in the stationary body 12. The PCB 190 may also be coupled to a phototransistor 194 with a rotary encoder for measuring motor turns. In aspects of the disclosure, the rotary encoder is a quadrature encoder that measures both speed and direction of the drive shaft of the motor 70.

The controller can include any suitable electrical components for operating the disclosed surgical stapling device or components thereof. The controller may include any type of computing device, computational circuit, or any type of processor or processing circuit capable of executing a series of instructions that are stored in memory. The controller may include multiple processors and/or multicore central processing units (CPUs) and may include any type of processor, such as a microprocessor, digital signal processor, microcontroller, programmable logic device (PLD), field programmable gate array (FPGA), or the like. The controller may also include a memory to store data and/or instructions that, when executed by the one or more processors, cause the one or more processors to perform one or more methods and/or algorithms.

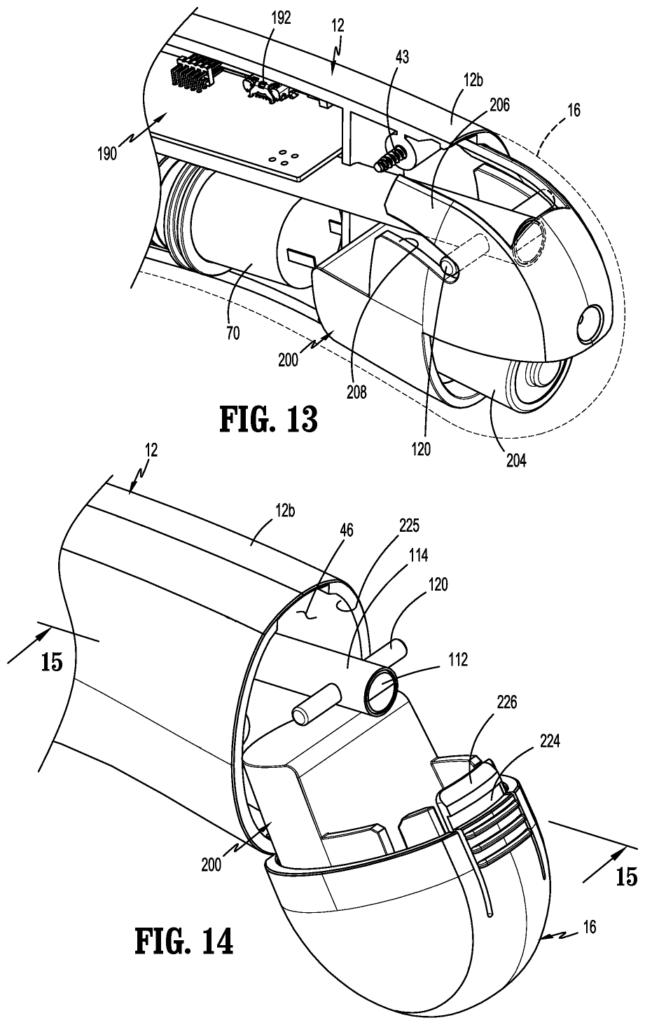

FIGS. 13-16 illustrate the proximal portion 12b of the stationary body 12 and the battery cover 16. The battery cover 16 supports a battery shell 200 that defines a cavity 202 (FIG. 16) for receiving a battery 204. The battery shell 200 includes a guide portion 206 (FIG. 13) that defines a pair of guide slots 208 that receive transverse pin 120 supported on the proximal end of the release tube 114 to guide the battery cover 16 as the battery cover is pivoted from its open position towards its closed position. The battery shell 200 is secured within the battery cover 16 using, e.g., a screw 210 to secure the battery 204 within the battery cover 16. The battery shell 200 includes a sidewall 212 (FIG. 16) wall that defines a concavity 212a that receives a circular protrusion 214 formed on the proximal portion 12b of the stationary body 12 of the handle assembly 10 to pivotably secure the battery cover 16 to the proximal portion 12b of the stationary body 12.

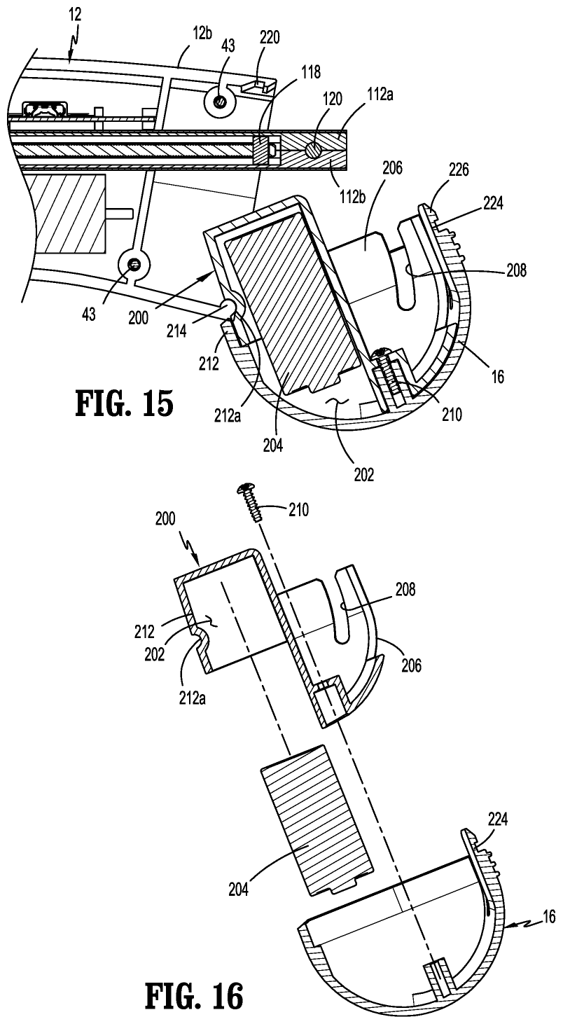

The proximal portion 12b of the stationary body 12 defines a recess 220 (FIG. 15) that is located at a position diametrically opposed to the circular protrusion 214. The battery cover 16 includes a flexible tab 224 that includes a tapered stop 226 that is received in the recess 220 in snap-fit fashion when the battery cover 16 is moved to the closed position to lock the battery cover 16 in the closed position. The flexible tab 224 can be pressed inwardly to remove the tapered stop 226 from within the recess 220 to move the battery cover 16 to its open position. The proximal portion 12b of the stationary body 12 defines an opening 225 (FIG. 14).

FIG. 14 illustrates the battery cover 16 in the open position. In this position, the transverse pin 120 on the release tube 114 is positioned proximally of the stationary body 12 and is accessible to a clinician. As described above, if the powered handle assembly 10 becomes disabled, the battery cover 16 can be pivoted to the open position to allow a clinician to manually rotate the release tube 114 by gripping the transverse pin 120 and rotating the release tube 114 to rotate the drive rod 92 in relation to the firing nut gear 96 to retract or advance the firing rod assembly 86.

The disclosed handle assembly 10 and stapling device 2 places the actuators and the articulation knob for actuating and articulating an end effector of the surgical device in close relation to each other to allow for single handed operation of the surgical device. The in-line handle assembly 10 is received in the palm of a clinician's hands and allows a clinician to rotate the end effector 300 (FIG. 1) of the surgical device by simply rotating the clinician's wrist thus obviating the need for additional rotation mechanisms. The battery 204 is received in a removable compartment in the battery cover 16 to allow for removal and recycling of the battery following use. The manual release tube 214 and pin 120 allows the firing rod assembly 86 to be manually retracted in emergency situations.

Persons skilled in the art will understand that the devices and methods specifically described herein and illustrated in the accompanying drawings are non-limiting exemplary aspects of the disclosure. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described aspects of the disclosure. Accordingly, the disclosure is not to be limited by what has been particularly shown and described, except as indicated by the appended claims.

1. (canceled)

2. A surgical device comprising:
a handle assembly including a stationary body, a distal body portion, and an articulation assembly, the distal body portion extending from the stationary body and defining a longitudinal axis, the articulation assembly including an articulation knob, an articulation nut, an articulation screw, and an articulation link, the articulation knob coupled to the articulation nut and rotatable about the longitudinal axis to rotate the articulation nut about the longitudinal axis, the articulation nut coupled to the articulation screw such that rotation of the articulation nut causes longitudinal movement of the articulation screw along the longitudinal axis, the articulation link coupled to the articulation screw such that longitudinal movement of the articulation screw causes longitudinal movement of the articulation link; and
an end effector pivotably supported on the distal body portion about an articulation axis that is transverse to the longitudinal axis, the articulation link coupled to the end effector to translate longitudinal movement of the articulation link to pivotable movement of the end effector about the articulation axis.

3. The surgical device of claim 2, wherein the articulation knob is coupled to the articulation nut by a pin.

4. The surgical device of claim 2, wherein the stationary body defines an annular recess and the articulation nut includes a flange that is received within the annular recess to facilitate rotation of the articulation nut in relation to the stationary body.

5. The surgical device of claim 2, wherein the articulation nut defines a threaded bore and the articulation screw includes an outer threaded surface that is received within the threaded bore of the articulation nut.

6. The surgical device of claim 2, wherein the articulation knob defines internal slots and the articulation nut includes longitudinal ribs that are received within the internal slots to couple the articulation nut to the articulation knob.

7. The surgical device of claim 2, wherein the articulation link is secured to the articulation screw by a pin.

8. The surgical device of claim 2, wherein the distal body portion of the handle assembly defines a longitudinal recess, and the articulation link is slidably received within the longitudinal recess.

9. The surgical device of claim 2, wherein the end effector forms part of a reload assembly, the reload assembly coupled to the distal body portion of the handle assembly.

10. The surgical device of claim 2, wherein the end effector includes an anvil assembly and a cartridge assembly, the end effector movable between an open position and a clamped position.

11. A handle assembly comprising:
a stationary body;
a distal body portion extending from the stationary body and defining a longitudinal axis; and
an articulation assembly including an articulation knob, an articulation nut, an articulation screw, and an articulation link, the articulation knob coupled to the articulation nut and rotatable about the longitudinal axis to rotate the articulation nut about the longitudinal axis, the articulation nut coupled to the articulation screw such that rotation of the articulation nut causes longitudinal movement of the articulation screw along the longitudinal axis, the articulation link coupled to the articulation screw such that longitudinal movement of the articulation screw causes longitudinal movement of the articulation link.

12. The handle assembly of claim 11, wherein the articulation knob is coupled to the articulation nut by a pin.

13. The handle assembly of claim 11, wherein the stationary body defines an annular recess and the articulation nut includes a flange that is received within the annular recess to facilitate rotation of the articulation nut in relation to the stationary body.

14. The handle assembly of claim 11, wherein the articulation nut defines a threaded bore and the articulation screw includes an outer threaded surface that is received within the threaded bore of the articulation nut.

15. The handle assembly of claim 11, wherein the articulation knob defines internal slots and the articulation nut includes longitudinal ribs that are received within the internal slots to couple the articulation nut to the articulation knob.

16. The handle assembly of claim 11, wherein the articulation link is secured to the articulation screw by a pin.

17. The handle assembly of claim 11, wherein the distal body portion of the handle assembly defines a longitudinal recess, and the articulation link is slidably received within the longitudinal recess.

18. An articulation assembly comprising:
an articulation knob, an articulation nut, an articulation screw, and an articulation link, the articulation nut defining a through bore and having internal threads, the articulation screw defining a longitudinal axis, the articulation knob coupled to the articulation nut and rotatable about the longitudinal axis to rotate the articulation nut about the longitudinal axis, the articulation nut including an outer threaded surface, the outer threaded surface of the articulation screw coupled to the internal threads of the articulation nut such that rotation of the articulation nut causes longitudinal movement of the articulation screw along the longitudinal axis, the articulation link coupled to the articulation screw such that longitudinal movement of the articulation screw causes longitudinal movement of the articulation link.

19. The articulation assembly of claim 18, wherein the articulation knob is coupled to the articulation nut by a pin.

20. The articulation assembly of claim 18, wherein the articulation knob defines internal slots and the articulation nut includes longitudinal ribs that are received within the internal slots to couple the articulation nut to the articulation knob.

21. The articulation assembly of claim 18, wherein the articulation nut includes a proximal portion having an annular flange.
